(19) United States
(12) Patent Application Publication
(10) Pub. No.: US 2014/0005249 A1
(43) Pub. Date: Jan. 2, 2014

(54) NKX2.2 INHIBITORS AS DRUGS

(71) Applicants: Jean-Philippe Hugnot, Montpellier (FR); Pierre-Olivier Guichet, Montpellier (FR); Marisa Teigell, Montpellier (FR); Ivan Bieche, Suresnes (FR); Rosette Lidereau, Gennevilliers (FR); Dominique Joubert, Sete (FR); Luc Bauchet, Clapiers (FR); Valerie Rigau, Mauguio (FR)

(72) Inventors: Jean-Philippe Hugnot, Montpellier (FR); Pierre-Olivier Guichet, Montpellier (FR); Marisa Teigell, Montpellier (FR); Ivan Bieche, Suresnes (FR); Rosette Lidereau, Gennevilliers (FR); Dominique Joubert, Sete (FR); Luc Bauchet, Clapiers (FR); Valerie Rigau, Mauguio (FR)

(73) Assignees: UNIVERSITE MONTPELLIER 2 SCIENCES ET TECHNIQUES, Montpellier Cedex 5 (FR); CENTRE NATIONAL DE LA RECHERCHE SCIENTIFIQUE, Paris Cedex 16 (FR); CENTRE HOSPITALIER UNIVERSITAIRE DE MONTPELLIER, Montpellier Cedex 5 (FR); INSTITUT CURIE, Paris Cedex 5 (FR)

(21) Appl. No.: 13/811,461

(22) PCT Filed: Jul. 21, 2011
(86) PCT No.: PCT/EP2011/062578

§ 371 Date: Apr. 9, 2013

(30) Foreign Application Priority Data
Jul. 21, 2010 (EP) 10305805.3

(51) Int. Cl. C12N 15/113; A61K 45/06

(52) U.S. Cl. CPC C12N 15/113; A61K 45/06
USPC 514/44 A; 514/44 R

(57) ABSTRACT

The present invention relates to NKX2.2 inhibitors such as shRNAs for treating pathologies.

The present invention relates to NKX2.2 inhibitors as drugs.

Cancer stem cells (CSCs) are cancer cells (found within tumors or hematological cancers) that possess characteristics associated with normal stem cells, specifically the ability to give rise to all cell types found in determined cancer sample.

CSCs may generate tumors through the stem cell processes of self-renewal and differentiation into multiple cell types.

CSCs have been identified initially in leukaemia sample wherein an isolated subpopulation of leukaemic cells that express a specific surface marker CD34, but lacks the CD38 marker are capable of initiating tumors in NOD/SCID mice that is histologically similar to the donor.

The existence of leukaemic stem cells prompted further research into other types of cancer. CSCs have recently been identified in several solid tumors, including cancers of the breast, brain, colon, ovary, pancreas and prostate.

The efficacy of cancer treatments is measured by the reduction of the tumor mass. However, since CSCs form a very small proportion of the tumor, they may not necessarily be targeted by the treatment. CSCs cells are proposed to persist in tumors as a distinct population and cause relapse and metastasis by giving rise to new tumors.

Therefore, there is a need to specifically eradicate cancer stem cells in order to limit relapse of tumors after remission, following antitumor therapies.

Gliomas are primitive tumours of the CNS which are derived from tumorigenesis of cells of the glial lineage (astrocytes and oligodendrocytes) (Louis, 2006, Annu Rev Pathol 1, 97-117; Behin, 2003, Lancet 361, 323-331). They are the most frequent brain tumours with an incidence of 1/20 000/inhabitants/year (3000 new cases in France, 15 000 news cases in US per year) (Bondy, 2008, Cancer. 2008 Oct. 1; 113(7 Suppl):1953-68; Bauchet 2007, J. Neurooncol. 2007 September; 84(2):189-99). These tumors are aggressive, highly invasive and neurologically destructive.

Glioma are divided in two main categories:
High grade gliomas (grade III-IV according to WHO classification, Louis, 2007, Acta Neuropathol. 2007 August; 114(2):97-109), which are mostly represented by multiform glioblastomas (GBM). These tumours contain highly proliferating cells and are associated with a very poor prognosis.
Low grade gliomas (WHO grade II glioma, G2G), which growth slowly but which ineluctably evolves to anaplasia within 5-10 years.

One important feature of gliomas is their diffuse aspect due to migration and infiltration of the parenchyma from which deterioration will occur. There is currently no curative treatment for these tumors and despite maximum treatment efforts, median survival of patients diagnosed with GBM ranges from 9 to 12 months, a statistic that has changed very little in decades.

GBM are the most common glioma in humans (Kleihues 2000, Cancer. 2000 Jun. 15; Maher, 2001, Genes Dev. 2001 Jun. 1; 15(11):1311-33) and can evolve from low grade glioma (secondary GBM) or develop de novo (primary GBM). Like all cancers, GBM share a relatively restricted set of characteristics crucial to their phenotype: proliferation in the absence of external growth stimuli, avoidance of apoptosis and no limits to replication, escape from both external growth-suppressive forces and the immune response, formation of new blood vessels and the ability to invade normal tissues (Hanahan and Weinberg 2000, Cell. 100(1):57-70). Furthermore, despite their striking heterogeneity, common alterations in specific cellular signal transduction pathways occur within most GBMs (Louis, 2006, Annu Rev Pathol 1, 97-117).

GBM may be derived from transformation of differentiated cells or alternatively of adult stem/progenitor cells (Dai 2003, Cancer J 9, 72-81; Holland, 2001, Curr Opin Neurol 14, 683-688).

Indeed, GBMs contain 1-20% of cancer stem cells which grow on non adherent substrates to generate clonal expansion called neurospheres. The latter are multipotential and generate astrocytes and neuronal-like cells upon differentiation on adhesive substrate, These cancer stem cells appear to be more tumorigenic than the rest of tumoral cells when grafted in immunocompromised animals. In addition, these cells seem to be more chemo- and radio-resistant than the other tumoral cells.

As a consequence, new glioma drugs or treatments have to be found to specifically eradicate these cells.

Tumoral stem cells, as the non tumoral stem cells, reside in special vascular niches (Gilbertson, 2007, Nat Rev Cancer 7, 733-736) which provide high level of canonical stem cell signallings such as Wnt, Notch or SHH (Ischenko, 2008, Curr Med. Chem. 2008; 15(30):3171-84). These pathways maintain the cells in an undifferentiated state and contribute to their self-renewal.

In addition, it is now well documented that GBM stem cells rely on a special set of genes (for instance Sox2, Olig2, Bmi1 . . . ) to maintain a high level of self-renewal. These genes could be considered as potential targets to specifically eliminate these cells.

The number of new molecules specifically developed to cure gliomas is very low.

Treatments of the proliferative tumoral cells transiently reduce tumor progression. However, a relapse occurs, due to the persistence of GBM stem cells.

One possible approach is to differentiate these CSCs into post-mitotic cells so as to turn off the proliferation program, and therefore limiting tumor growth.

This approach has very satisfying results in Acute Promyelocytic leukemia (APL) treated with retinoic acid and/or arsenic.

An alternative possibility is to inactivate one or several stem cell signallings with specific drugs targeting these pathways (Ischenko, 2008, Curr Med. Chem. 2008; 15(30):3171-84).

Last, one can also consider the possibility of targeting key stem cell genes to eradicate the source of the tumor.

During embryogenesis, glial differentiation appears in dorsal and ventral area of neural tube. This process is directly controlled by sonic hedgehog (SHH) and involved transcription factors OLIG2 and NKX2.2. NKX2.2 has emerged as a key regulator of oligodendrocyte differentiation.

It regulates the differentiation and/or maturation of oligodendrocyte progenitors, and is required for mature beta-cell function and islet structure, as demonstrated by the functional invalidation in mice (Sussel et al. 1998. Development 125(12), p: 2213-2221).

It has been also demonstrated that NKX2.2 is a marker for oncogenic transformation of Ewing's sarcoma where its presence correlates with a poor prognosis, (Smith R, et al. (2006) Cancer Cell; 9(5):405-16, Owen L A, et al. (2008), PLoS One. 3(4):e1965, Cheung I Y, et al. (2007) Clin Cancer Res. 13(23):6978-83).

So, NKX2.2 appears to be a good candidate in order to eradicate stem cells, and consequently cancer stem cells of gliomas.

Surprisingly, NKX2.2 biallelic invalidation in mice does not modify glial differentiation, but only impairs pancreatic beta cell differentiation (Sussel et al. 1998. Development 125(12), p: 2213-2221). NKX2.2−/− mice born without neural deficiencies, but die rapidly with a severe diabetes due to the absence terminal differentiation of pancreatic β cells.

In Ewing's sarcoma tumors, it has been demonstrated that NKX2.2 participates to the EWS-Fli oncogenic pathway, and that its inhibition repress tumor progression. As a consequence, US 2008280844 patent application proposes the inhibition of NKX2.2 in order to treat cancer. However, this document only demonstrates that NKX2.2 inhibition limit the growth of Ewing's sarcoma cells, but stay silent about the risk of relapse of the tumor.

So the need of an efficient drug able to completely and efficiently eradicate cancer cells, and cancer stem cells, remains.

Therefore, one aim of the invention is to provide a new efficient drug for treating pathologies, including cancer.

Another aim of the invention is to provide an efficient therapy for treating glioma tumors, without risk of relapse, by targeting the NKX2.2 gene.

The disclosure relates to a product inhibiting
the expression of the gene coding for the NKX2.2 protein, and/or
the activity of the NKX2.2 protein,
as cell death inducing drug, in particular as apoptotic drug.

The invention relates to a product inhibiting
the expression of the gene coding for the NKX2.2 protein, and/or
the activity of the NKX2.2 protein,
for its use for inducing apoptosis of tumoral cells.

The present invention is based on the unexpected observation made by the Inventors that the suppression of the expression of the gene coding NKX2.2 protein, or the inactivation of the NKX2.2 protein, induces cell death.

“Compounds” or “product” are equally used in the invention to define inhibitor of the expression of the gene coding for the NKX2.2 protein, and/or

the activity of the NKX2.2 protein.

By convention, in the invention, the name of proteins is capitalized (e.g. NKX2.2) and the corresponding gene coding for said protein is represented by slanting characters (e.g. NKX2.2).

The invention encompasses the use of all the compounds which have an activity that either inhibits the expression of the gene coding for NKX2.2, or the activity of the NKX2.2 protein, or both, i.e. inhibits the expression of the gene coding for NKX2.2 and the activity of the NKX2.2 protein.

By “inhibiting the expression of the gene coding for the NKX2.2 protein” it is meant in the invention that the mechanisms
of transcription of the gene coding for NKX2.2,
of maturation or stability of the messenger produced by the transcription of the gene coding for NKX2.2, or
of spicing of said messenger,
is reduced from about 50% to 99%, or abolished; by the use of the inhibitors according to the invention, in such that the NKX2.2 protein expression is reduced from 50% to 99%, or abolished.

It is also meant that the inhibitors according to the invention are specific of said gene coding for NKX2.2 protein, or homologous proteins, but have no effects on the expression of one or more genes coding for proteins different from NKX2.2 proteins.

By “inhibiting the activity of the NKX2.2 protein”, it is meant in the invention that the function of the NKX2.2., i.e. gene expression regulation, is reduced from 50 to 99%, or abolished by the use of the inhibitors.

As for the inhibition of the gene expression, the inhibition of the activity of NKX2.2 is also specific of the NKX2.2 protein, or homologous proteins. Thus, proteins different from NKX2.2 protein, or homologous proteins thereof, have an activity not affected by the inhibitors according to the invention.

The compounds used according to the invention kill cells.

There are many possibilities to kill cells: by inducing programmed cellular death (also called apoptosis), by inducing necrosis, or by inducing autophagy.

Autophagy, or autophagocytosis, is a catabolic process involving the degradation of a cellular own components through the lysosomal machinery. It is a tightly-regulated process that plays a normal part in cell growth, development, and homeostasis, helping to maintain a balance between the synthesis, degradation, and subsequent recycling of cellular products. It is a major mechanism by which a starving cell reallocates nutrients from unnecessary processes to more-essential processes.

Apoptosis is the process of programmed cell death that may occur in multicellular organisms. Biochemical events lead to characteristic cell changes (morphology) and death. These changes include blebbing, loss of cell membrane asymmetry and attachment, cell shrinkage, nuclear fragmentation, chromatin condensation, and chromosomal DNA fragmentation. Necrosis corresponds to the premature death of cells and living tissue. Necrosis is caused by factors external to the cell or tissue, such as infection, toxins, or trauma. This is in contrast to apoptosis, which is a naturally occurring cause of cellular death. While apoptosis often provides beneficial effects to the organism, necrosis is almost always detrimental and can be fatal.

The inhibitors or compounds according to the invention are able to kill cells rapidly and efficiently, as illustrated in the Example section.

An advantageous embodiment of the invention relates to the compounds used as defined above as apoptotic drug, able to kill tumoral cells, preferably cancer stem cells.

“Apoptotic drug” according to the invention defines a drug having properties to induce programmed cell death of cells treated with said drug.

Apoptosis can be easily measured by general protocols known in the art.

These protocols include, for instance:
annexin V detection at the cell surface, for instance by using flow cytometry,
caspase activation measurement, such as Caspase 3 activation,
DNA fragmentation measurement, for instance by TUNEL method,
Cytochrome C measurement.

It is also possible to measure apoptosis by flow cytometry (FACS) by measuring the DNA content, in particular by quantifying the population of cells having a DNA content lower than the DNA content of a diploid cell (sub G1 population).

The skilled person is able also to measure cell apoptosis by other well described methods disclosed in the art.

In an advantageous embodiment, the invention relates to a product used as defined above, wherein said NKX2.2 protein comprises or consists of
the amino acid sequence SEQ ID NO:1, or
any amino acid sequence having at least 85% of identity with the amino acid sequence SEQ ID NO:1, preferably any amino acid sequence having at least 90% of identity with the amino acid sequence SEQ ID NO:1.

The proteins according to the invention having at least 85% of identity to the amino acid sequence SEQ ID NO:1 harbor the same, or substantially the same, activity than the activity of the protein comprising or consisting in the amino acid sequence SEQ ID NO: 1, but differ in their sequence.

The human NKX2.2 protein is referenced in databases under the accession number NP_{—}002500 (Seq_Ref)

In another advantageous embodiment, the invention relates to a product used as defined above, wherein said gene coding for the NKX2.2 protein comprises or consists of
the nucleic acid sequence SEQ ID NO: 2, or
any nucleic acid molecule having at least 75%, preferably at least 85%, more preferably at least 95% of homology with the nucleic acid sequence SEQ ID NO: 2.

The above mentioned sequence homology can be, for instance, the consequence of the genetic code degeneracy, well known by the skilled person in the art.

The human NKX2.2 gene (mRNA) is referenced in databases under the accession number NM_{—}002509 (Seq_Ref)

In another advantageous embodiment, the invention relates to a product used as mentioned above, wherein said product inhibiting the expression of the gene coding for the NKX2.2 protein is chosen among
at least one siRNA,
at least one miRNA,
at least one shRNA, and
at least one antisens nucleic acid molecule,
or a combination of the above.

In one more advantageous embodiment, the invention relates to a product used as mentioned above, wherein said product inhibiting the expression of the gene coding for the NKX2.2 protein is selected from the group consisting of: at least one siRNA, and at least one shRNA.

siRNA or shRNA according to the invention inhibit NKX2.2 gene expression by RNA interference mechanism.

RNA interference is a highly conserved biological mechanism inducing specific repression of genes by specifically destroying mRNA, or inhibition translation of said RNA.

In 1998, Fire et al[Fire zt al., Nature. 1998 Feb. 19; 391(6669):806-11] demonstrated that a double-stranded is produced in cells by a Class III RNA endonuclease, the DICER complex, and small inhibiting double-stranded RNA (siRNA) of about 19 to 28 nucleotides are produced.

Incorporated to the enzymatic <<RNA—Induced Silencing Complex>> RISC complex, said siRNA are deshybridized and can therefore hybridize with the complementary sequence contained in mRNA. The “captured” mRNA is then destroyed, or its translation by ribosomal particles is inhibited.

Small hairpin RiboNucleic Acid—shRNA are double-stranded molecules comprising both the sense and the antisense strand of a siRNA, said sense and antisense strands being linked by a linker. These molecules form a hairpin, and the linker is eliminated to allow the liberation of a siRNA.

In still another advantageous embodiment, the invention relates to a product used as previously defined, wherein said siRNA comprises or consists of one of the following nucleic acid sequences:

SEQ ID NO: 3
CUUCUACGACAGCAGCGACAA,
SEQ ID NO: 4
UUGUCGCUGCUGUCGUAGAAG,
SEQ ID NO: 5
CAAACCAUGUCACGCGCUCAA,
SEQ ID NO: 6
UUGAGCGCGUGACAUGGUUUG,
SEQ ID NO: 7
CCUGCCGGACACCAACGAUGA,
SEQ ID NO: 8
UCAUCGUUGGUGUCCGGCAGG,
SEQ ID NO: 9
CCAUGCCUCUCCUUCUGAA,
and
SEQ ID NO: 10
UUCAGAAGGAGAGGCAUGG,
in association with their complementary
sequence.

The complementary sequence of the nucleic acid molecule comprising or consisting of SEQ ID NO: 3 comprises or consists of the sequence SEQ ID NO: 4. The complementary sequence of the nucleic acid molecule comprising or consisting of SEQ ID NO: 5 comprises or consists of the sequence SEQ ID NO: 6. The complementary sequence of the nucleic acid molecule comprising or consisting of SEQ ID NO: 7 comprises or consists of the sequence SEQ ID NO: 8. The complementary sequence of the nucleic acid molecule comprising or consisting of SEQ ID NO: 9 comprises or consists of the sequence SEQ ID NO: 10.

Thus, most advantageous siRNA according to the invention are one of the following siRNA:
siRNA comprising a sens strand comprising or consisting in SEQ ID NO: 3 and its complementary sequence, or antisens strand, comprising or consisting of SEQ ID NO: 4,
siRNA comprising a sens strand comprising or consisting in SEQ ID NO: 5 and its complementary sequence, or antisens strand, comprising or consisting of SEQ ID NO: 6,
siRNA comprising a sens strand comprising or consisting in SEQ ID NO: 7 and its complementary sequence, or antisens strand, comprising or consisting of SEQ ID NO: 8, and
siRNA comprising a sens strand comprising or consisting in SEQ ID NO: 9 and its complementary sequence, or antisens strand, comprising or consisting of SEQ ID NO: 10.

The above siRNA can also be modified by addition of compounds stabilizing siRNA structure.

For instance, the above siRNA contain, in their 3′-end a dinucleotide: a dithymidine (TT). Therefore, the siRNA according to the invention comprise one of the following sequences:

SEQ ID NO: 11
CUUCUACGACAGCAGCGACAATT,
SEQ ID NO: 12
UUGUCGCUGCUGUCGUAGAAGTT,
SEQ ID NO: 13
CAAACCAUGUCACGCGCUCAATT,
SEQ ID NO: 14
UUGAGCGCGUGACAUGGUUUGTT,
SEQ ID NO: 15
CCUGCCGGACACCAACGAUGATT,
SEQ ID NO: 16
UCAUCGUUGGUGUCCGGCAGGTT,
SEQ ID NO: 17
CCAUGCCUCUCCUUCUGAATT,
and
SEQ ID NO: 18
UUCAGAAGGAGAGGCAUGGTT.

Thus, most advantageous siRNA according to the invention are one of the following siRNA:
siRNA comprising a sens strand comprising or consisting in SEQ ID NO: 11 and its complementary sequence, or antisens strand, comprising or consisting of SEQ ID NO: 12,
siRNA comprising a sens strand comprising or consisting in SEQ ID NO: 13 and its complementary sequence, or antisens strand, comprising or consisting of SEQ ID NO: 14,
siRNA comprising a sens strand comprising or consisting in SEQ ID NO: 15 and its complementary sequence, or antisens strand, comprising or consisting of SEQ ID NO: 16, and
siRNA comprising a sens strand comprising or consisting in SEQ ID NO: 17 and its complementary sequence, or antisens strand, comprising or consisting of SEQ ID NO: 18.

In one another advantageous embodiment, the invention relates to a product used as mentioned above, wherein said shRNA comprises or consists of one of the following nucleic acid molecules:
a nucleic acid molecule comprising or being constituted by the sequence SEQ ID NO: 3 followed by the sequence SEQ ID NO: 4, the 3′-end of SEQ ID NO:3 being linked to the 5′-end of SEQ ID NO: 4 by a linker.
a nucleic acid molecule comprising or being constituted by the sequence SEQ ID NO: 5 followed by the sequence SEQ ID NO: 6, the 3′-end of SEQ ID NO:5 being linked to the 5′-end of SEQ ID NO: 6 by a linker.
a nucleic acid molecule comprising or being constituted by the sequence SEQ ID NO: 7 followed by the sequence SEQ ID NO: 8, the 3′-end of SEQ ID NO:7 being linked to the 5′-end of SEQ ID NO: 8 by a linker.
a nucleic acid molecule comprising or being constituted by the sequence SEQ ID NO: 9 followed by the sequence SEQ ID NO: 10, the 3′-end of SEQ ID NO:9 being linked to the 5′-end of SEQ ID NO: 10 by a linker.

The linker according to the invention can be chosen among the following linkers

1) UUCAAGAGA
(Brummelkamp, T.R., 2002 Science. 296(5567):
550-3),
2) AAGUUCUCU
(Promega),
3) UUUGUGUAG
(Scherr, M., Curr Med Chem. 2003 Feb; 10(3):
245-56.),
(SEQ ID NO: 19)
4) CUUCCUGUCA
(Schwarz D.S., 2003 Cell. 115(2): 199-208.),
and
5) CUCGAG.

In one other advantageous embodiment, the invention relates to a product used as mentioned above, wherein said shRNA is in the form of a DNA molecule comprising or consisting of one of the following molecule:
a nucleic acid molecule comprising or being constituted by the sequence SEQ ID NO: 20 followed by the sequence SEQ ID NO: 21, the 3′-end of SEQ ID NO: 20 being linked to the 5′-end of SEQ ID NO: 21 by a linker.
a nucleic acid molecule comprising or being constituted by the sequence SEQ ID NO: 22 followed by the sequence SEQ ID NO: 23, the 3′-end of SEQ ID NO: 22 being linked to the 5′-end of SEQ ID NO: 23 by a linker.
a nucleic acid molecule comprising or being constituted by the sequence SEQ ID NO: 24 followed by the sequence SEQ ID NO: 25, the 3′-end of SEQ ID NO: 24 being linked to the 5′-end of SEQ ID NO: 25 by a linker.
a nucleic acid molecule comprising or being constituted by the sequence SEQ ID NO: 26 followed by the sequence SEQ ID NO: 27, the 3′-end of SEQ ID NO: 26 being linked to the 5′-end of SEQ ID NO: 27 by a linker.

The linker according to the invention can be chosen among the following linkers

1) TTCAAGAGA
(Brummelkamp, T.R., 2002 Science. 296(5567):
550-3),
2) AAGTTCTCT
(Promega),
3) TTTGTGTAG
(Scherr, M., Curr Med Chem. 2003 Feb; 10(3):
245-56.),
(SEQ ID NO: 28)
4) CTTCCTGTCA
(Schwarz D.S., 2003 Cell. 115(2): 199-208.),
and
5) CTCGAG.

In one particular embodiment, the shRNA used according to the invention comprise or consist of one of the following sequences:

SEQ ID NO: 29
CCGGCTTCTACGACAGCAGCGACAACTCGAGTTGTCGCTG
CTGTCGTAGAAGTTTTT
SEQ ID NO: 30
CCGGCAAACCATGTCACGCGCTCAACTCGAGTTGAGCGCG
TGACATGGTTTGTTTTT
SEQ ID NO: 31
CCGGCCTGCCGGACACCAACGATGACTCGAGTCATCGTTGG
TGTCCGGCAGGTTTTT
SEQ ID NO: 32
CCGGCCATGCCTCTCCTTCTGAATTcaagagaTTCAGAAGG
AGAGGCATGGTTTTTG

In one advantageous embodiment, the invention relates to the nucleic acid molecule comprising or consisting of the sequence SEQ ID NO: 32, as apoptotic drug, for its use for inducing apoptosis of tumoral cells.

According to another advantageous embodiment, the invention relates to a product used as defined above, wherein said shRNA is comprised in a vector, said vector comprising nucleic acid sequences allowing the expression of said shRNA.

The above mentioned sequences allowing the expression of said shRNA are in particular promoter used by the RNA polymerase III, such as U6 promoter, H1 promoter, or any other polymerase III promoters used in the art. These vectors could be for instance pTRIPZ or pGIPZ lentivectors (Openbiosystems Company).

In one another advantageous embodiment, the invention relates to a product used as mentioned above, wherein said compound inhibiting the activity NKX2.2 protein is chosen among:
at least a protein specifically interacting with said NKX2.2 protein, said protein being preferably an antibody or an aptamer (Bouchard, 2010 Annu Rev Pharmacol Toxicol. 50:237-57) or an inhibiting form of a NKX2.2 partner protein, such as OLIG2, Groucho co-repressors 1 to 4 (GRG1-4) or mSIN3A proteins, or a fragment thereof,
at least a dominant negative form of said NKX2.2 protein, and
at least a DNA molecule interacting with said NKX2.2 protein.

Another possibility to inhibit NKX2.2, in order to provide the product according to the invention, consists to enforce the expression of proteins or nucleic acid molecules interfering with NKX2.2 activity.

For instance, by enforcing the expression of proteins regulating the activity of NKX2.2, it is possible to decrease or to abolish its activity.

Therefore, according to the invention, it is possible to use proteins such as OLIG2, Groucho co-repressor (Grg1-4) proteins or mSIN3A proteins, or fragments thereof of said proteins; said fragment retaining their ability to interact and to modulate NKX2.2 activity.

By “dominant negative form of NKX2.2 protein”, the invention defines a NKX2.2 modified protein interfering with the NKX2.2 protein. For instance, a dominant negative form can be constituted by the DNA binding domain of NKX2.2 fused to a transactivating domain of a transcription factor activating the transcription (VP16 for instance). This fusion will activate the transcription of NKX2.2 target genes instead of repressing their expression, and therefore interfering with the natural function of NKX2.2. A dominant negative form could theoretically be constituted by a NKX2.2 protein deleted of the DNA binding domain. By competing with the full length protein for the association with the NKX2.2 partners but by not binding to DNA this deleted form will decrease the number of functional NKX2.2 transcriptional complexes.

The above mentioned DNA binding domain of NKX2.2 is located from the amino acid residue at position 135 to the amino acid residue at position 185 of the amino acid sequence SEQ ID NO:1, and consists to the amino acid sequence SEQ ID NO: 39.

One another possibility to inhibit NKX2.2 protein consists to inhibit its ability to interact with specific DNA sequences. Thus, by over expressing DNA target sequence of NKX2.2, said NKX2.2 is “sequestrated” and become unable to specifically regulate its target genes.

The sequence that can be used for “sequestrating” NKX2.2 correspond to the consensus target sequence having the following nucleic acid sequence: ((T(C/T)AAGT(G/A)(G/C)TT) (SEQ ID NO: 40)

In one another advantageous embodiment, the invention relates to a product used as defined above, wherein:
OLIG2 protein comprises or consists in the amino acid sequence SEQ ID NO: 33,
Groucho co-repressors 1 to 4 comprises or consists of one of the amino acid sequence SEQ ID NO: 34 to 37, and
mSIN3A comprises or consists in the amino acid sequence SEQ ID NO: 38.

The invention also relates to a composition comprising:

1. a product as defined above, and

2. at least one antitumoral agent,
for its use for inducing apoptosis of tumoral cells.

The above composition can be associated with a pharmaceutically acceptable carrier. The appropriate pharmaceutically acceptable carrier is determined by the skilled person.

For instance, if the composition used according to the invention contains proteins, said composition can be in a form of liposome, microsphere carriers, or the protein can be in a form of fusion protein with VIH TAT protein or Protein Transduction Domain (PTD) of viral proteins.

The above mentioned carriers are such that they allow the delivery to, and the entry into, the target cell of the protein contained in the composition according to the invention.

In particular, pharmaceutically acceptable carrier allows crossing the blood brain barrier (BBB).

For instance, nucleic acid molecules are encapsulated in a 100 nm pegylated liposome and conjugated with receptor specific targeting monoclonal antibodies can cross the BBB and target tumoral cells (Pardridge, 2007, Pharm Res.24(9):1733-44).

Dosage of the active substance depends on the administration route, and can be easily determined by a skilled person. The composition used according to the invention can be administered by intravenous route, sub-cutaneous route, systemic route, or can be administered locally by infiltration, or per os.

The composition used according to the invention can be administered at a dosage from about 0.001 g/kg/day to about 0.1 g/kg/day, according to the administration route.

In particular, the compositions used according to the invention may be administered at a dosage from about 0.05 to about 5 g/day in adults, or from about 0.01 to about 1 g/day for children.

The composition used according to the invention may be a pharmaceutical composition, in association with a pharmaceutically acceptable carrier.

In an advantageous embodiment, the pharmaceutical composition used according to the invention contains at least a compound as previously defined in a form of the pharmaceutically acceptable salts known to a person skilled in the art, such as sodium salts, ammonium salts, calcium salts, magnesium salts, potassium salts, acetate salts, carbonate salts, citrate salts, chloride salts, sulphate salts, amino chlorhydate salts, borhydrate salts, phosphate salts, dihydrogenophosphate salts, succinate salts, citrate salts, tartrate salts, lactate salts, mandelate salts, methane sulfonate salts (mesylate) or p-toluene sulfonate salts (tosylate).

In one advantageous embodiment, the invention relates to a pharmaceutical composition comprising the nucleic acid molecule comprising or consisting of the sequence SEQ ID NO: 32, as apoptotic drug, in association with a pharmaceutically acceptable carrier, for its use for inducing apoptosis of tumoral cells.

In one advantageous embodiment, the invention relates to a pharmaceutical composition used as defined above, further comprising at least one antitumoral agent.

“Antitumoral agent” means in the invention any compounds having an activity which inhibit cell proliferation, or enhance apoptosis of tumour cells and correspond to compounds or a drugs commonly used for treating cancer in the frame of chemotherapy. These compounds are called chemotherapeutic agents. The skilled person can easily determine from the pathology which antitumor compound can be added to the compounds according to the invention.

The majority of antitumoral agents can be divided into alkylating agents, antimetabolites, anthracyclines, plant alkaloids, topoisomerase inhibitors. These agents commonly interfere with cell division and DNA replication, and therefore limiting the multiplication of cancer cells.

Some advantageous antitumoral agents according to the invention are cisplatin, vincristin, vinblastin, taxanes compounds or ectoposides.

For instance, one of the following treatments: Temozolomide, Cisplatine, BCNU (Carmustine), CCNU (Lomustine) and more recently Campto (Iritonecan-CPT 11)-Avastin (Bevacizumab) can be used with the pharmaceutical composition according to the invention.

In one advantageous embodiment, the invention relates to a pharmaceutical composition used as defined above, wherein said product and said antitumoral agent are used in a simultaneous, separate or sequential manner.

In one other advantageous embodiment, the invention relates to a pharmaceutical composition used as defined above, for its use for the treatment of central nervous system (CNS) tumors expressing NKX2.2 and gastro-entero-pancreatic neuroendocrine (GEP NE) tumors that express this gene (Wang, 2009, Endocr Relat Cancer. 16(1):267-79).

In another advantageous embodiment, the invention relates to a pharmaceutical composition comprising at least one product inhibiting,
the expression of the gene coding for the NKX2.2 protein, and/or
the activity of the NKX2.2 protein.
for its use for the treatment of central nervous system (CNS) tumors expressing NKX2.2 protein and NKX2.2+ gastro-entero-pancreatic neuroendocrine (GEP NE) tumors (Wang, 2009, Endocr Relat Cancer. 16(1):267-79).

In one another advantageous embodiment, the invention relates to a pharmaceutical composition, for its use as defined above, comprising at least one product as defined above,

wherein said NKX2.2 protein comprises or consists of
the amino acid sequence SEQ ID NO:1, or
any amino acid sequence having at least 85% of identity with the amino acid sequence SEQ ID NO:1, preferably any amino acid sequence having at least 90% of identity with the amino acid sequence SEQ ID NO:1, or
wherein said gene coding for the NKX2.2 protein comprises or consists of
the nucleic acid sequence SEQ ID NO: 2, or
any nucleic acid molecule having at least 75%, preferably at least 85%, more preferably at least 95% of homology with the nucleic acid sequence SEQ ID NO: 2.

In one another advantageous embodiment, the invention relates to a pharmaceutical composition, for its use as defined above, comprising at least one product as defined above, wherein said product inhibiting the expression of the gene coding for the NKX2.2 protein is chosen among
at least one sRNA, preferably comprising one of the sequences SEQ ID NO: 3 to 18, and the corresponding complementary sequence as defined above
at least one miRNA,
at least one shRNA, preferably comprising one of the sequences SEQ ID NO: 29 to 31, and the corresponding complementary sequence as defined above, and
at least one antisens nucleic acid molecule,
or a combination of the above.

In one advantageous embodiment, the invention relates to a pharmaceutical composition used as defined above, wherein said CNS tumors are chosen among the group consisting of: grade II, grade III and grade IV glioma according to The 2007 WHO classification of tumours of the central nervous system (Louis, Acta Neuropathol. 2007 August; 114(2):97-109).

In one advantageous embodiment, the invention relates to a pharmaceutical composition used as defined above, wherein said neuroendocrines tumors are chosen among the group consisting of primary and metastatic Gastro-entero-pancreatic neuroendocrine tumors, in particular said neuroendocrines tumors expressing NKX2.2 proteins.

The invention also relates to a method for treating central nervous system (CNS) tumors and gastro-entero-pancreatic neuroendocrine (GEP NE) tumors expressing NKX2.2, in a patient in a need thereof, comprising the administration of a pharmaceutically effective amount of a pharmaceutical composition comprising at least a product as defined above, said product inhibiting
the expression of the gene coding for the NKX2.2 protein, and/or
the activity of the NKX2.2 protein.

In one another advantageous embodiment, the invention relates to a method as defined above,

wherein said NKX2.2 protein comprises or consists of
the amino acid sequence SEQ ID NO:1, or
any amino acid sequence having at least 85% of identity with the amino acid sequence SEQ ID NO:1, preferably any amino acid sequence having at least 90% of identity with the amino acid sequence SEQ ID NO:1, or
wherein said gene coding for the NKX2.2 protein comprises or consists of
the nucleic acid sequence SEQ ID NO: 2, or
any nucleic acid molecule having at least 75%, preferably at least 85%, more preferably at least 95% of homology with the nucleic acid sequence SEQ ID NO: 2.

In one another advantageous embodiment, the invention relates to a method as defined above, wherein said product inhibiting the expression of the gene coding for the NKX2.2 protein is chosen among
at least one sRNA, preferably comprising one of the sequences SEQ ID NO: 3 to 18, and the corresponding complementary sequence as defined above
at least one miRNA,
at least one shRNA, preferably comprising one of the sequences SEQ ID NO: 29 to 31, and the corresponding complementary sequence as defined above, and
at least one antisens nucleic acid molecule,
or a combination of the above.

In one advantageous embodiment, the invention relates to a method for treating central nervous system (CNS) tumors and gastro-entero-pancreatic neuroendocrine (GEP NE) tumors expressing NKX2.2, in a patient in a need thereof, comprising the administration of a pharmaceutically effective amount of a nucleic acid molecule comprising or consisting of the sequence SEQ ID NO: 32, as apoptotic drug.

The present invention is illustrated by the following examples and the following 11 figures.

### FIGURES

FIGS. 1A-L represent immunofluorescence of GBM stem cells transfected with a GFP expressing plasmid used as a reporter gene and NKX2.2 shRNA 32 or non relevant shRNA

FIGS. 1A-D represent DNA staining using DAPI.

FIGS. 1E-H represent transfected cells expressing bright GFP

FIGS. 1I-L represent NKX2.2 staining by using immunofluorescence anti NKX2.2 antibody (arrows).

Cells represented in FIGS. 1A, E, I, B, F, and J have been cotransfected with non relevant shRNA (luciferase) and GFP plasmids.

Cells represented in FIGS. 1C, G, K, D, H, and L have been cotransfected with NKX2.2 shRNA 32 and GFP plasmids, and show no remaining NKX2.2 nuclear staining.

FIG. 2 corresponds to a graph representing the number of neurospheres obtained 4 days after transfection with non relevant (luciferase; first column) or NKX2.2 (SEQ ID NO 32) shRNA (second column). Y-axis represent the number of neurosphere per well. ** represent a p<0.01, according to Mann-Whitney tests.

FIG. 3 corresponds to a graph representing the cell number obtained after 4 days following transfection with the indicated shRNA. Scramble (first column) and luciferase (second column) correspond to non relevant shRNA, and shRNA 29, 30, 31, 32 are against NKX2.2 mRNA (corresponding respectively to SEQ ID NO: 29, 30, 31 and 32; respectively third, fourth, fifth and sixth column). ** represent a p<0.01, according to Mann-Whitney tests. Y-axis represents the cell number per well.

FIGS. 4A and B represent direct microscopic observation of GBM stem cells cultured as neurospheres. Scale bar represents 100 μm.

FIG. 4A represents a neurosphere culture from cells transfected with non relevant shRNA (shRNA luc).

FIG. 4B represents a neurosphere culture from cells transfected with NKX2.2 shRNA n° 32 (SEQ ID NO: 32).

FIG. 5 corresponds to a graph representing the number of GBM stem cells cultured on adherent surface, 4 days after transfection with a non relevant (shRNA luciferase; first column) or NKX2.2 (shRNA 32, SEQ ID NO 32; second column) shRNA. Y-axis represent the number of cell per well. ** represent a p<0.01, according to Mann-Whitney tests.

FIG. 6 corresponds to a graph representing the fold increase of the number of apoptotic cells detected by cleaved caspase 3 immunodetection in GBM stem cell cultures after transfection with anti NKX2.2 shRNA 32 vs Luciferase shRNA. Y-axis represents the fold increase of the number of apoptotic cells.

FIG. 7 represents the expression of NKX2.2 gene in glioma tumors. The columns represent the fold increase of NKX2.2 gene in Grade II (second column), Ill (third column), IV glioma (fourth column) compared to NKX2.2 gene expression in non tumoral brain (first column). Grade IV glioma (GBM) shows an almost 5-fold overexpression of NKX2.2 compared to non tumoral brain. Non parametric Kruskal and Wallis H tests were used to compare gene expression medians. Error bars represent standard error of mean (sem). *** represent p<0.001 and * represent p<1.05. Y-axis represents the fold increase compared to non tumoral cells

FIG. 8 represents immunofluorescence detection of NKX2.2 protein in a glioblastoma tumor section. Note the large proportion of cells expressing Nkx2.2 (white staining).

FIG. 9 represents immunofluorescence detection of nuclear NKX2.2 protein (white staining) in GBM stem cells cultured as neurospheres.

FIG. 10 represents histograms indicating the number of cells after treatment with control shRNA (dark grey columns), or with shRNA 3 or 4 (light grey and white columns respectively) in 3 cells lines: Gli4 (3 first columns), Gli5 (columns 4-6) and Gli7 (columns 7-9). Y axis represents the number of cells, expressed as % of control cells.

FIG. 11 represents histograms indicating the fold change in the mRNA expression of anti apoptotic (BCL2; first column) and pro apoptotic, (BAX and BAK; respectively second and third columns) genes induced 24 h after expression of Nkx2.2 shRNA4 or control shRNA.

Y-axis represents the fold change of mRNA expression (shRNA NKX2.2/sh RNA control)

### EXAMPLES

### Example 1

### NKX2.2 is an Essential Gene for GBM Cell Growth and Survival

Cellular Model

The importance of the NKX2.2 gene in GBM stem cells was investigated using four cancer stem cell lines (Gli4F11, Gli4, Gli5, Gli7) which was derived from patients diagnosed with a high grade glioma tumor (GBM, grade IV according to WHO classification).
1. These lines grow and self-renew in non adherent conditions and are able to form clonal neurospheres when seeded at one cell in 96 wells dish.
2. These lines have an abnormal caryotype with hallmarks of GBM (chromosome 7 gain).
3. These lines express typical cancer stem cell markers (nestin, CD133, CD15) and contains a side population (Hoescht exclusion).
4. These lines are multipotential and generates GFAP+, Map2ab+ and GalC+ cells after differentiation.
5. These lines generate highly infiltrating high grade tumors in NOD/SCID mice. After 4 months, the whole brain is invaded by tumoral cells which remain proliferative.
6. These lines strongly expresses NKX2.2 at the mRNA and protein level in vitro, and in xenotransplanted animals.

Cell Culture

Gli4F11, Gli4, Gli5, Gli7 cells are cultured in serum-free DMEM/F12 media supplemented with non vitamin A-B27 (2%) and N2 (1%) serum-replacement media (Invitrogen), FGF2, and EGF2 (10 ng/ml each, Peproteck), Heparine (2 μg/ml, Sigma), Ciprofloxaxine (2 μg/ml, Euromedex), Gentamycine (10 μg/ml, Fisher), fungin (10 μg/ml, Cayla) and fungizone (0.25 μg/ml, Fisher). The cell are passaged classically using complete dissociation with trypsine/EDTA 0.25% for 3′ followed by trypsin inactivation with trypsin inhibitor (Sigma, T9003). Cells are either grown as free-floating neurospheres on non-adherent substrate coated flasks (poly-2-hydroxyethylmethacrylate, poly HEMA from Sigma) or adherent substrate coated dishes (poly-D-lysine (25 μg/ml) and laminin (2 μg/cm², Sigma)).

Effect of Anti NKX2.2 shRNA on Gli4F11 Growth and Neurosphere Formation

shRNA:

Controls (scramble or non silencing luciferase shRNA) and NKX2.2 shRNAs cloned in pLKO plasmids were purchased from Openbiosystems (shRNA 29, 30, 31) or kindly provided by Dr Lessnick's lab (Huntsman Cancer Institute, Salk Lake City, USA) (shRNA 32). The shRNA plasmids were purified from bacteria using a Quiagen kit (endotoxin-free quality) according to the manufacturer procedure.

Transfection:

shRNA plasmids were transfected using a nucleofection method with an Amaxa apparatus and neural stem cell kit (Lonza-Amaxa, kit VPG-1004).

Prior to nucleofection, Gli4F11 cells were dissociated and resuspended at 5 millions per 100 μl in nucleofection buffer.

A mixture of plasmids (10 μg total) containing the shRNA plasmid against NKX2-2 and a plasmid for selecting transfected cells (pCMV-EGFP (clonetech)) (ratio 3:1) was added.

For control conditions, either a shRNA against luciferase or a scramble shRNA were used.

Cells were transfected using Amaxa nucleofection program number A-033 and rapidly resuspended in 500 μl of media at 37° C. and seeded in 75 cm2 flask containing 15 ml of complete media.

Sorting of Transfected Cells

Twenty four hours after transfection, the cells were completely dissociated and GFP+ cells were purified using cytometry on a Aria BD cytometer.

Results

NKX2.2 expression in GBM and GBM stem cells.

The NKX2.2 mRNA level was measured in tumors by QPCR (Light cycler Roche) on 20 samples of each glioma grade (II, III, IV) and compared to NKX2.2 expression measured in 20 non tumoral brain samples. FIG. 7 indicates that NKX2.2 expression is highly correlated to glioma malignity. Grade IV glioma (GBM) shows an almost 5-fold overexpression of NKX2.2 compared to non tumoral brain. Significances: *** (p<0.001), * (p≦0.05). (Non parametric Kruskal and Wallis H)

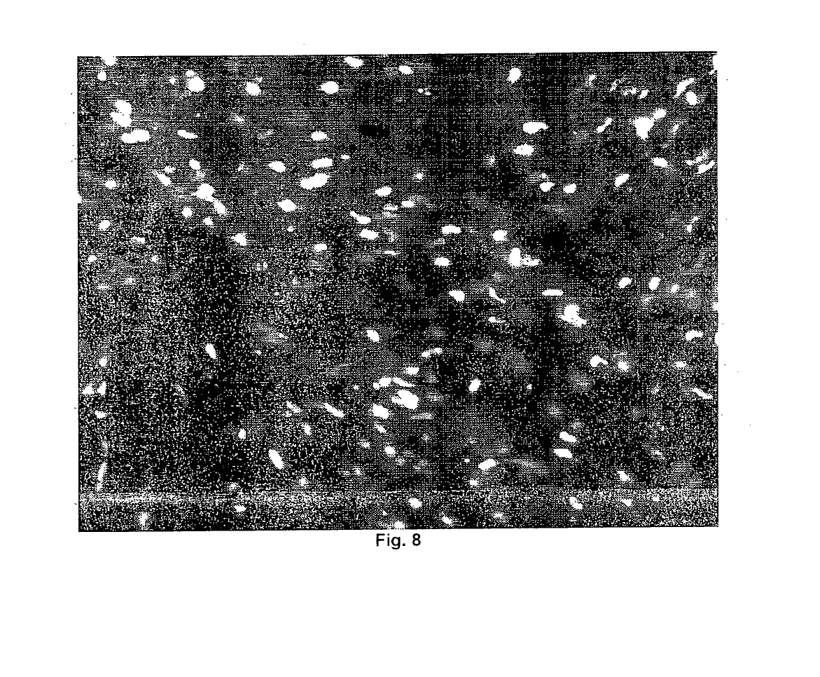

Accordingly, FIG. 8 shows that NKX2.2 protein is highly expressed by a large proportion of cells in a GBM tumor section. NKX2.2 is detected here by classical immunofluorescence (white nuclear staining).

FIG. 9 shows that the NKX2.2 protein (white nuclear staining), is highly expressed by most of the cancer stem cells cultured as neurospheres. Nkx2.2 is detected here by classical immunofluorescence (white nuclear staining).

Inhibition of NKX2.2 mRNA and Protein Expression by Anti NKX2.2 shRNA

Decrease of NKX2 mRNA Level

Twenty four hours after transfection, the cells were dissociated and GFP⁺ cells were purified as described above. Extraction of total mRNA was performed using RNeasy mini kit (Qiagen) according to the manufacturer procedure. QPCR (Light Cycler Roche) was used to determine the level of NKX2.2 RNA using PO RNA as a internal control for normalisation. Expression levels are represented in the following table 1. This table shows that the shRNA against NKX2.2 (shRNA 29 and 32) reduce the level of NKX2.2 mRNA by 46 and 62% respectively as compared to control shRNA.

TABLE 1
NKX2-2

| | CT | /P0 | normalized |
|---|---|---|---|
| Contrôle Scramble | 27.36 | 0.009 | 1.00 |
| Contrôle Luciferase | 27.42 | 0.009 | 0.96 |
| shRNA 29 | 28.34 | 0.005 | 0.54 |
| sh RNA 32 | 28.85 | 0.004 | 0.38 |

CT refers to number of PCR cycle. The column “/P0” represents the quantification of NKX2.2 relative to the P0 RNA level. Normalization represents the values obtained with the anti NKX2.2 shRNA relative to control shRNA.

Decrease of NKX2 Protein Level

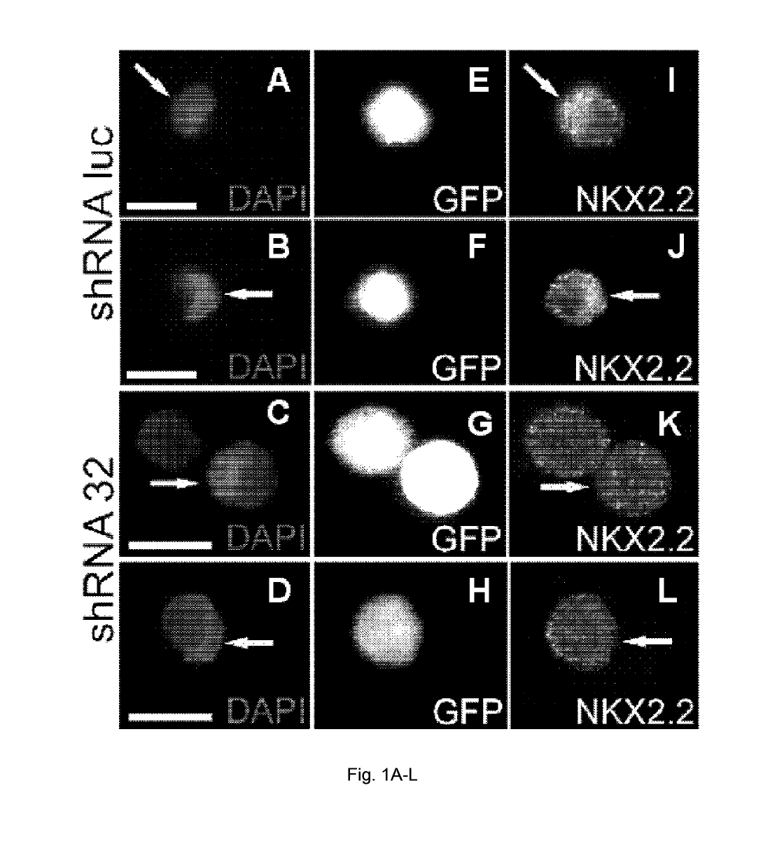

Twenty four hours after transfection, the cells were dissociated and GFP+ cells purified as described above were seeded on coverslips coated with poly-ornithin, centrifugated 15 minutes at 1000 rpm and fixed with paraformaldehyde 4%. Expression of NKX2.2 in the cells was determined by immunofluorescence with a monoclonal anti NKX2.2 antibody (Developmental Studies Hybridoma Bank, number 74.5A5). The decrease of NKX2.2 protein in NKX2.2 shRNA compared to luciferase shRNA transfected cells was analysed using AxiolmagerZ1/Apotome microscope. Immunofluorescences are shown in FIGS. 1A-L.

FIGS. 1A, 1B, 1C, 1D show cell nuclei stained with DAPI. FIGS. 1E, 1F, 1G, 1H show bright transfected cells expressing GFP. FIGS. 1I, 1J, 1K, 1L show nuclear NKX2.2 protein detected with anti NKX2.2 antibody. FIGS. 1K, 1L indicate that the nuclear NKX2.2 staining (arrows) almost disappear in cells transfected with NKX2.2 shRNA compared to control transfected cells (arrows in FIGS. 1I and 1J).

Assessment of Neurosphere Formation

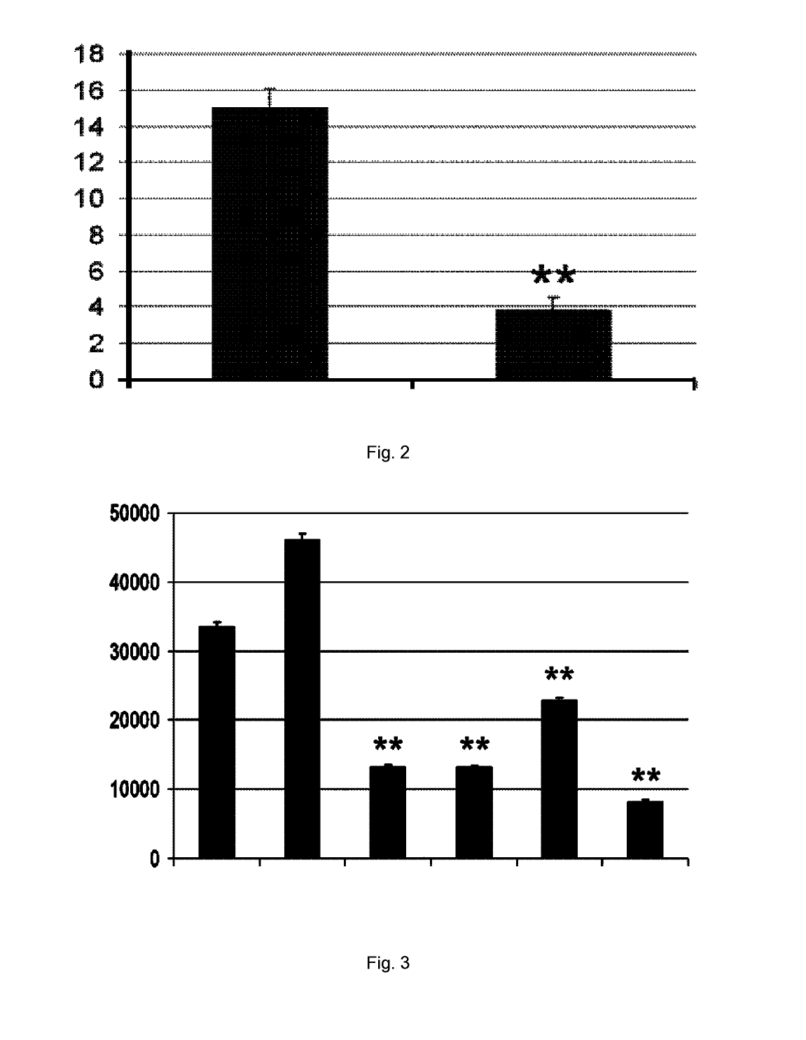

One important property of normal and GBM stem cells is their ability to form neurospheres when plated at clonal density on non adherent dishes. To evaluate whether NKX2.2 shRNA could affect this property, after transfection and cell sorting, the cells were seeded at 1 cell/μl in 25 cm2 flask (5 ml of media) coated with poly HEMA. After 7 days without moving the flask (so as to reduce the possibility of aggregation and the formation of non clonal neurospheres), the number of the neurospheres were determined by using visual scanning of the entire 25 cm2 flasks and a graduated ocular (Nikon cell culture microscope). Quantification of the neurosphere number is represented on FIG. 2. Compared to control shRNA (shRNA luciferase), NKX2.2 shRNA 32 induces an almost 4 fold reduction in the number of GBM neurospheres. Significance: ** (p<0.01), Mann-Whitney tests.

Assessment of Growth

This was assessed using non adherent or adherent conditions.

1—Transfected cells sorted by GFP expression were seeded at 30 000 cells per wells in 24-wells plates (6 wells per condition, 1 ml of media) coated with poly-2-hydroxyethylmethacrylate. Four days after, the cells were completely dissociated by directly adding 300 μl of Trypsin 2.5% (sigma, 4799) into the well. After 10 minutes at 37° C., the cells were triturated and their number was counted using Z2 coulter counter (Beckman) using a 10-20 μm window.

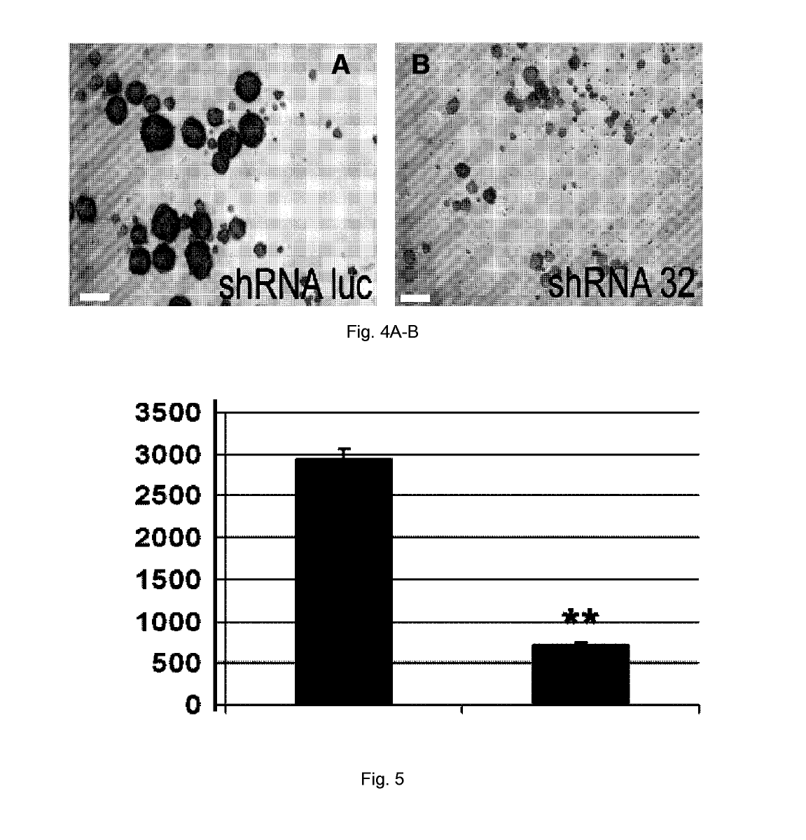

FIG. 3 shows that compared to control shRNAs (Scramble and Luciferase), any of the four shRNA against NKX2.2 (shRNA 29, 30, 31, 32) induces a reduction of the cell number, ranging from a 1.4 reduction for shRNA31 to almost 4 fold for shRNA 32. FIG. 4 shows photographs of cultures 5 days after transfection with control shRNA (shRNA luc) or anti NKX2.2 shRNA (shRNA 32). Significance: ** (p<0.01), Mann-Whitney tests.
For adherent conditions, sorted cells were seeded on pDL/laminin coated plates at 30 000 cells per wells in 24-wells plate (6 wells per conditions, 1 ml of media). The same protocol used for non adherent cell was used to determine the cell number after 4 days of growth. FIG. 5 shows that compared to control shRNA (Luciferase), shRNA against NKX2.2 (shRNA 32) induces an almost 4 fold reduction of the cell number. Significance: ** (p<0.01), Mann-Whitney tests.

Effect of Anti NKX2.2 shRNA on Gli4F11 Apoptosis

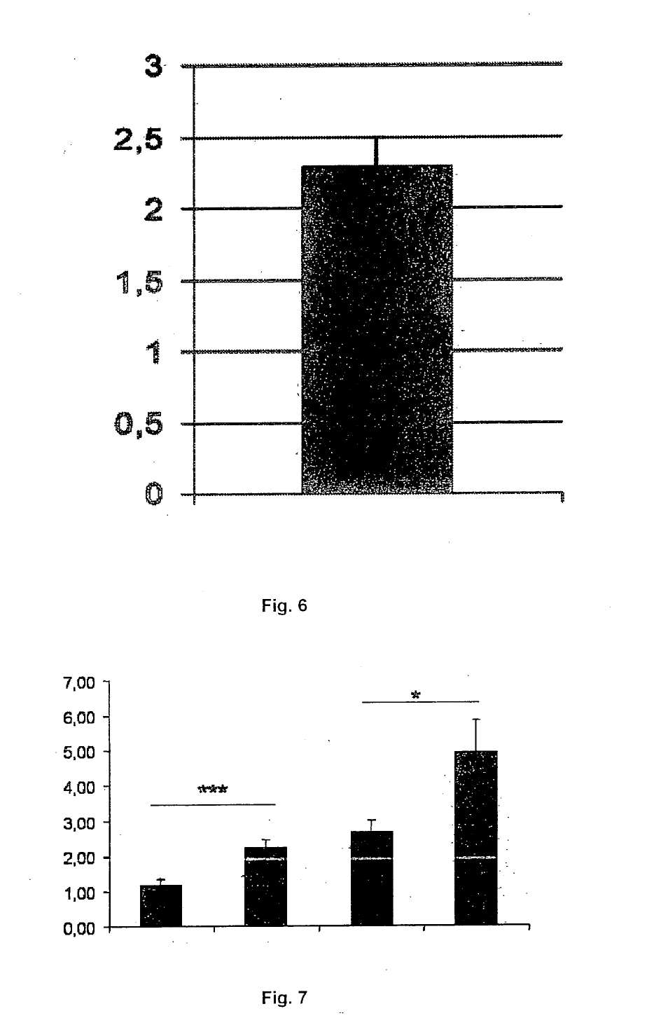

Apoptotic cells induced by NKX2.2 shRNA were detected by immunofluorescence against cleaved caspase 3. Twenty four hours after the transfection with the shRNA plasmid, the cells were dissociated and the transfected cells were purified on the basis of GFP expression as described above. Cells were seeded on coverslips coated with pDL/Laminin, incubated 10 minutes at 37° C. then fixed with paraformaldehyde 4%. Immunofluorescence was classically performed using an anti cleaved caspase 3 antibody (Cell Signaling, 9661). The number of apoptotic cells in anti NKX2.2 shRNA and anti luciferase transfected cells was determined using manual counting of the entire coverslips with AxiolmagerZ1/Apotome microscope (Zeiss). FIG. 6 shows that the anti NKX2.2 shRNA 32 induces a 2.2 fold increase of apoptotic cells compared to cells transfected with the control shRNA (luc) (n=3).

### Example 2

### Effect of Overexpression of Proteins Inhibiting NKX2.2 Activity

In this approach, the cells are transfected by a reporter gene (pCMV-GFP) together with a plasmid encoding a dominant negative form of NKX2.2 or a protein reducing the NKX2.2 activity such as an aptamer (Bouchard, 2010, Annu Rev Pharmacol Toxicol. 2010; 50:237-57). In parallel, cells are transfected by an adequate control plasmid (empty vector). After sorting of transfected cells, these are plated in GBM stem cell media as previously described. After 3-5 days of growth, the total cell number and the number of apoptotic cells are determined by the methods described above.

### Example 3

### Effect of Overexpression of DNA Target Sequence for Inhibiting NKX2.2 Activity

In this approach, the cells are transfected by a reporter gene (pCMV-GFP) together with a plasmid encoding several copies of the NKX2.2 binding sites so as to compete for the endogenous sites and sequestrate NKX2.2 protein. After sorting of transfected cells, these are plated in grown in GBM stem cell media as previously described. After 3-5 days of growth, the total cell number and the number of apoptotic cells are assayed by the methods described above.

### Example 4

### Inhibition of Polyclonal Glioma Cell Growth by Targeting Nkx2.2 by RNA Interference

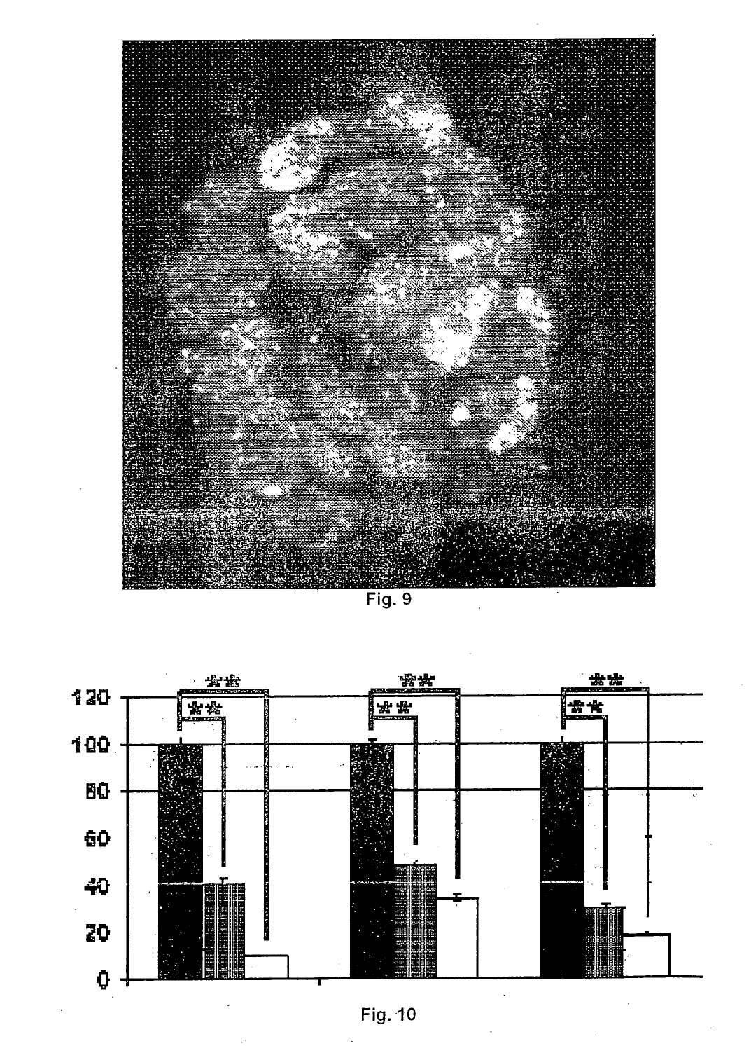

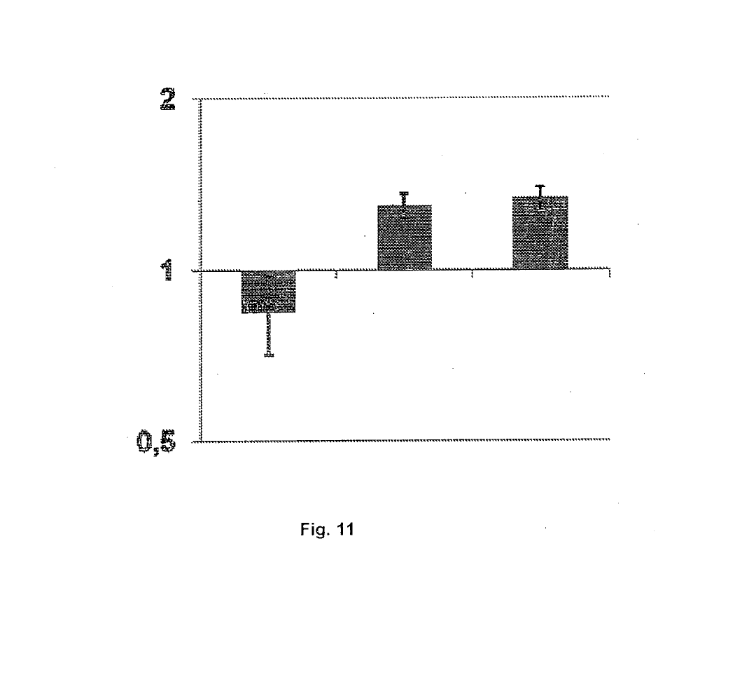

In addition to the clonal Gli4F11 cell line, the role of Nkx2.2 was explored in 3 others glioma cultures. The Inventors confirmed their results by using the primary polyclonal Gli4 culture from which Gli4F11 was derived and by deriving two new cultures (Gli5 and Gli7) from two patients affected by Gb. Like Gli4F11, these three lines presented a phenotype reminiscent of oligodendrocyte progenitors as evidenced by the expression of Ascl1, NG2/CSPG4, Nkx2.2, Olig1/2 and PDGFRa markers. These cultures are multipotent, have abnormal karyotypes and form highly infiltrative Olig2⁺ Nkx2.2⁺ tumors similar to Gli4F11 when grafted in immunocompromised mice. In these 3 cultures, transfection of shRNA3 and 4, drastically reduced the cell number after 5 days which was associated with an overt cell death, as shown in FIG. 10.

### Example 5

### Influence of the Loss of Function of Nkx2.2 on the Expression of Pro Apoptotic and Anti Apoptotic Genes in Gli4F11 Cell Lines

Gli4F11 cells were transiently transfected with anti Nkx2.2 shRNA 4 or control shRNA plasmids then 24 h later, QPCR was performed for the proapoptotic (Bak, Bax) and the anti apoptotic (Bcl2) genes. As illustrated on FIG. 11, compared to cells transfected with control shRNA the downregulation of Nkx2.2 caused an increase of Bak and Bax mRNAs and in contrast a decrease of Bcl2 transcripts (n=3 experiments). The observed increase in the ratio of pro/anti apoptotic genes induced by the shRNA anti Nkx2.2 will lead to cell death.

1-12. (canceled)

13. A method for treating central nervous system (CNS) tumors and gastro-entero-pancreatic neuroendocrine (GEP NE) tumors expressing NKX2.2, in a patient in a need thereof, comprising the administration of a pharmaceutically effective amount of a product inhibiting
a. the expression of the gene coding for the NKX2.2 protein, and/or
b. the activity of the NKX2.2 protein.

14. The method according to claim 13, wherein said NKX2.2 protein comprises or consists of:
the amino acid sequence SEQ ID NO:1, or
any amino acid sequence having at least 85% of identity with the amino acid sequence SEQ ID NO:1, preferably any amino acid sequence having at least 90% of identity with the amino acid sequence SEQ ID NO:1.

15. The method according to claim 13, wherein said gene coding for the NKX2.2 protein comprises or consists of
the nucleic acid sequence SEQ ID NO: 2, or
any nucleic acid molecule having at least 75%, preferably at least 85%, more preferably at least 95% of homology with the nucleic acid sequence SEQ ID NO: 2.

16. The method according to claim 13, wherein said product inhibiting the expression of the gene coding for the NKX2.2 protein is chosen among
at least one siRNA
at least one miRNA
at least one shRNA, and
at least one antisens nucleic acid molecule,
or a combination of the above.

17. The method according to claim 16, wherein said siRNA comprises or consists of one of the following nucleic acid sequences: SEQ ID NO: 3 to 10.

18. The method according to claim 16, wherein said shRNA comprises or consists of one of the following nucleic acid sequences: SEQ ID NO: 29, SEQ ID NO: 30, SEQ ID NO: 31 and SEQ ID NO: 32.

19. The method according to claim 18, wherein said shRNA is comprised in a vector, said vector comprising nucleic acid sequences allowing the expression of said shRNA.

20. The method according to claim 13, wherein said CNS tumors are chosen among the group consisting of: grade II, III and grade IV glioma according to The 2007 World Health Organisation classification of tumours of the central nervous system.

21. The method according to claim 13, wherein said neuroendocrinestumors are chosen among the group consisting of primary and metastatic gastro-entero-pancreatic neuroendocrine tumors.

22. The method according to claim 13, comprising the administration of a pharmaceutically effective amount of
a. a product inhibiting
i. the expression of the gene coding for the NKX2.2 protein, and/or
ii. the activity of the NKX2.2 protein,
and
at least one antitumoral agent.


SEQUENCE LISTING (40 sequences)

SEQ ID NO: 1 - protein, 273 aa, Homo sapiens
MSLTNTKTGFSVKDILDLPDTNDEEGSVAEGPEEENEGPEPAKRAGPLGQGALDAVQSLPLKNPFYDSSDNPYTRWLASTEGLQYSLHGLAAGAPPQDSSSKSPEPSADESPDNDKETPGGGGDAGKKRKRRVLFSKAQTYELERRFRQQRYLSAPEREHLASLIRLTPTQVKIWFQNHRYKMKRARAEKGMEVTPLPSPRRVAVPVLVRDGKPCHALKAQDLAAATFQAGIPFSAYSAQSLQHMQYNAQYSSASTPQYPTAHPLVQAQQWTW

SEQ ID NO: 2 - DNA, 2092 bp, Homo sapiens
gcggccgccggagcccgagctgacgccgccttggcacccctcctggagttagaaactaaggccggggcccgcggcgctcggcgcgcaggccgcccggcttcctgcgtccatttccgcgtgctttcaaagaagacagagagaggcactgggttgggcttcatttttttcctccccatccccagtttctttctctttttaaaaataataattatcccaataattaaagccaattcccccctcccctcccccagtccctccccccaactcccccctcccccgcccgccggggcaggggagcgccacgaattgaccaagtgaagctacaactttgcgacataaattttggggtctcgaaccatgtcgctgaccaacacaaagacggggttttcggtcaaggacatcttagacctgccggacaccaacgatgaggagggctctgtggccgaaggtccggaggaagagaacgaggggcccgagccagccaagagggccgggccgctggggcagggcgccctggacgcggtgcagagcctgcccctgaagaaccccttctacgacagcagcgacaacccgtacacgcgctggctggccagcaccgagggccttcagtactccctgcacggtctggctgccggggcgccccctcaggactcaagctccaagtccccggagccctcggccgacgagtcaccggacaatgacaaggagaccccgggcggcgggggggacgccggcaagaagcgaaagcggcgagtgcttttctccaaggcgcagacctacgagctggagcggcgctttcggcagcagcggtacctgtcggcgcccgagcgcgaacacctggccagcctcatccgcctcacgcccacgcaggtcaagatctggttccagaaccaccgctacaagatgaagcgcgcccgggccgagaaaggtatggaggtgacgcccctgccctcgccgcgccgggtggccgtgcccgtcttggtcagggacggcaaaccatgtcacgcgctcaaagcccaggacctggcagccgccaccttccaggcgggcattcccttttctgcctacagcgcgcagtcgctgcagcacatgcagtacaacgcccagtacagctcggccagcaccccccagtacccgacagcacaccccctggtccaggcccagcagtggacttggtgagcgccgccccaacgagactcgcggccccaggcccaggccccaccccggcggcggtggcggcgaggaggcctcggtccttatggtggttattattattattataattattattatggagtcgagttgactctcggctccactagggaggcgccgggaggttgcctgcgtctccttggagtggcagattccacccacccagctctgcccatgcctctccttctgaaccttgggagagggctgaactctacgccgtgtttacagaatgtttgcgcagcttcgcttctttgcctctccccggggggaccaaaccgtcccagcgttaatgtcgtcacttgaaaacgagaaaaagaccgaccccccacccctgctttcgtgcattttgtaaaatatgtttgtgtgagtagcgatattgtcagccgtcttctaaagcaagtggagaacactttaaaaatacagagaatttcttcctttttttaaaaaaaaataagaaaatgctaaatatttatggccatgtaaacgttctgacaactggtggcagatttcgcttttcgttgtaaatatcggtggtgattgttgccaaaatgaccttcaggaccggcctgtttcccgtctgggtccaactcctttctttgtggcttgtttgggtttgttttttgttttgtttttgtttttgcgttttcccctgctttcttcctttctctttttattttattgtgcaaacatttctcaaatatggaaaagaaaaccctgtaggcagggagccctctgccctgtcctccgggccttcagccccgaacttggagctcagctattcggcgcggttccccaacagcgccgggcgcagaaagctttcgattttttaaataagaattttaataaaaatcctgtgtttaaaaaagaaaaaaagaaaaaa

SEQ ID NO: 3 - RNA, 21 nt, Artificial Sequence
  derived from human NKX2.2
cuucuacgacagcagcgacaa

SEQ ID NO: 4 - RNA, 21 nt, Artificial Sequence
  derived from human NKX2.2
uugucgcugcugucguagaag

SEQ ID NO: 5 - RNA, 21 nt, Artificial Sequence
  derived from human NKX2.2
caaaccaugucacgcgcucaa

SEQ ID NO: 6 - RNA, 21 nt, Artificial Sequence
  derived from human NKX2.2
uugagcgcgugacaugguuug

SEQ ID NO: 7 - RNA, 21 nt, Artificial Sequence
  derived from human NKX2.2
ccugccggacaccaacgauga

SEQ ID NO: 8 - RNA, 21 nt, Artificial Sequence
  derived from human NKX2.2
ucaucguugguguccggcagg

SEQ ID NO: 9 - RNA, 19 nt, Artificial Sequence
  derived from human NKX2.2
ccaugccucuccuucugaa

SEQ ID NO: 10 - RNA, 19 nt, Artificial Sequence
  derived from human NKX2.2
uucagaaggagaggcaugg

SEQ ID NO: 11 - DNA, 23 bp, Artificial Sequence
  derived from human NKX2.2
cttctacgacagcagcgacaatt

SEQ ID NO: 12 - DNA, 23 bp, Artificial Sequence
  derived from human NKX2.2
ttgtcgctgctgtcgtagaagtt

SEQ ID NO: 13 - DNA, 23 bp, Artificial Sequence
  derived from human NKX2.2
caaaccatgtcacgcgctcaatt

SEQ ID NO: 14 - DNA, 23 bp, Artificial Sequence
  derived from human NKX2.2
ttgagcgcgtgacatggtttgtt

SEQ ID NO: 15 - DNA, 23 bp, Artificial Sequence
  derived from human NKX2.2
cctgccggacaccaacgatgatt

SEQ ID NO: 16 - DNA, 23 bp, Artificial Sequence
  derived from human NKX2.2
tcatcgttggtgtccggcaggtt

SEQ ID NO: 17 - DNA, 21 bp, Artificial Sequence
  derived from human NKX2.2
ccatgcctctccttctgaatt

SEQ ID NO: 18 - DNA, 21 bp, Artificial Sequence
  derived from human NKX2.2
ttcagaaggagaggcatggtt

SEQ ID NO: 19 - RNA, 10 nt, Artificial Sequence
  derived from human NKX2.2
cuuccuguca

SEQ ID NO: 20 - DNA, 21 bp, Artificial Sequence
  derived from human NKX2.2
cttctacgacagcagcgacaa

SEQ ID NO: 21 - DNA, 21 bp, Artificial Sequence
  derived from human NKX2.2
ttgtcgctgctgtcgtagaag

SEQ ID NO: 22 - DNA, 21 bp, Artificial Sequence
  derived from human NKX2.2
caaaccatgtcacgcgctcaa

SEQ ID NO: 23 - DNA, 21 bp, Artificial Sequence
  derived from human NKX2.2
ttgagcgcgtgacatggtttg

SEQ ID NO: 24 - DNA, 21 bp, Artificial Sequence
  derived from human NKX2.2
cctgccggacaccaacgatga

SEQ ID NO: 25 - DNA, 21 bp, Artificial Sequence
  derived from human NKX2.2
tcatcgttggtgtccggcagg

SEQ ID NO: 26 - DNA, 19 bp, Artificial Sequence
  derived from human NKX2.2
ccatgcctctccttctgaa

SEQ ID NO: 27 - DNA, 19 bp, Artificial Sequence
  derived from human NKX2.2
ttcagaaggagaggcatgg

SEQ ID NO: 28 - DNA, 10 bp, Artificial Sequence
  derived from human NKX2.2
cttcctgtca

SEQ ID NO: 29 - DNA, 57 bp, Artificial Sequence
  derived from human NKX2.2
ccggcttctacgacagcagcgacaactcgagttgtcgctgctgtcgtagaagttttt

SEQ ID NO: 30 - DNA, 57 bp, Artificial Sequence
  derived from human NKX2.2
ccggcaaaccatgtcacgcgctcaactcgagttgagcgcgtgacatggtttgttttt

SEQ ID NO: 31 - DNA, 57 bp, Artificial Sequence
  derived from human NKX2.2
ccggcctgccggacaccaacgatgactcgagtcatcgttggtgtccggcaggttttt

SEQ ID NO: 32 - DNA, 57 bp, Artificial Sequence
  derived from human NKX2.2
ccggccatgcctctccttctgaattcaagagattcagaaggagaggcatggtttttg

SEQ ID NO: 33 - protein, 323 aa, Homo sapiens
MDSDASLVSSRPSSPEPDDLFLPARSKGSSGSAFTGGTVSSSTPSDCPPELSAELRGAMGSAGAHPGDKLGGSGFKSSSSSTSSSTSSAAASSTKKDKKQMTEPELQQLRLKINSRERKRMHDLNIAMDGLREVMPYAHGPSVRKLSKIATLLLARNYILMLTNSLEEMKRLVSEIYGGHHAGFHPSACGGLAHSAPLPAATAHPAAAAHAAHHPAVHHPILPPAAAAAAAAAAAAAVSSASLPGSGLPSVGSIRPPHGLLKSPSAAAAAPLGGGGGGSGASGGFQHWGGMPCPCSMCQVPPPHHHVSAMGAGSLPRLTSDAK

SEQ ID NO: 34 - protein, 770 aa, Homo sapiens
MFPQSRHPTPHQAAGQPFKFTIPESLDRIKEEFQFLQAQYHSLKLECEKLASEKTEMQRHYVMYYEMSYGLNIEMHKQTEIAKRLNTICAQVIPFLSQEHQQQVAQAVERAKQVTMAELNAIIGQQQLQAQHLSHGHGPPVPLTPHPSGLQPPGIPPLGGSAGLLALSSALSGQSHLAIKDDKKHHDAEHHRDREPGTSNSLLVPDSLRGTDKRRNGPEFSNDIKKRKVDDKDSSHYDSDGDKSDDNLVVDVSNEDPSSPRASPAHSPRENGIDKNRLLKKDASSSPASTASSASSTSLKSKEMSLHEKASTPVLKSSTPTPRSDMPTPGTSATPGLRPGLGKPPAIDPLVNQAAAGLRTPLAVPGPYPAPFGMVPHAGMNGELTSPGAAYASLHNMSPQMSAAAAAAAVVAYGRSPMVGFDPPPHMRVPTIPPNLAGIPGGKPAYSFHVTADGQMQPVPFPPDALIGPGIPRHARQINTLNHGEVVCAVTISNPTRHVYTGGKGCVKVWDISHPGNKSPVSQLDCLNRDNYIRSCKLLPDGCTLIVGGEASTLSIWDLAAPTPRIKAELTSSAPACYALAISPDSKVCFSCCSDGNIAVWDLHNQTLVRQFQGHTDGASCIDISNDGTKLWTGGLDNTVRSWDLREGRQLQQHDFTSQIFSLGYCPTGEWLAVGMESSNVEVLHVNKPDKYQLHLHESCVLSLKFAYCGKWFVSTGKDNLLNAWRTPYGASIFQSKESSSVLSCDISVDDKYIVTGSGDKKATVYEVIY

SEQ ID NO: 35 - protein, 706 aa, Homo sapiens
MVQSRLTATSASQDSPASGLQTPLQSGQPFKFSILEICDRIKEEFQFLQAQYHSLKLECEKLASEKTEMQRHYVMYYEMSYGLNIEMHKQAEIVKRLSGICAQIIPFLTQEHQQQVLQAVERAKQVTVGELNSLIGQQQLQPLSHHAPPVPLTPRPAGLVGGSATGLLALSGALAAQAQLAAAVKEDRAGVEAEGSRVERAPSRSASPSPPESLVEEERPSGPGGGGKQRADEKEPSGPYESDEDKSDYNLVVDEDQPSEPPSPATTPCGKVPICIPARRDLVDSPASLASSLGSPLPRAKELILNDLPASTPASKSCDSSPPQDASTPGPSSASHLCQLAAKPAPSTDSVALRSPLTLSSPFTTSFSLGSHSTLNGDLSVPSSYVSLHLSPQVSSSVVYGRSPVMAFESHPHLRGSSVSSSLPSIPGGKPAYSFHVSADGQMQPVPFPSDALVGAGIPRHARQLHTLAHGEVVCAVTISGSTQHVYTGGKGCVKVWDVGQPGAKTPVAQLDCLNRDNYIRSCKLLPDGRSLIVGGEASTLSIWDLAAPTPRIKAELTSSAPACYALAVSPDAKVCFSCCSDGNIVVWDLQNQTMVRQFQGHTDGASCIDISDYGTRLWTGGLDNTVRCWDLREGRQLQQHDFSSQIFSLGHCPNQDWLAVGMESSNVEILHVRKPEKYQLHLHESCVLSLKFASCVQGVVLSPEL

SEQ ID NO: 36 - protein, 769 aa, Homo sapiens
MYPQGRHPAPHQPGQPGFKFTVAESCDRIKDEFQFLQAQYHSLKVEYDKLANEKTEMQRHYVMYYEMSYGLNIEMHKQTEIAKRLNTILAQIMPFLSQEHQQQVAQAVERAKQVTMTELNAIIGQQQLQAQHLSHATHGPPVQLPPHPSGLQPPGIPPVTGSSSGLLALGALGSQAHLTVKDEKNHHELDHRERESSANNSVSPSESLRASEKHRGSADYSMEAKKRKAEEKDSLSRYDSDGDKSDDLVVDVSNEDPATPRVSPAHSPPENGLDKARSLKKDAPTSPASVASSSSTPSSKTKDLGHNDKSSTPGLKSNTPTPRNDAPTPGTSTTPGLRSMPGKPPGMDPIASALRTPISITSSYAAPFAMMSHHEMNGSLTSPGAYAGLHNIPPQMSAAAAAAAAAYGRSPMVSFGAVGFDPHPPMRATGLPSSLASIPGGKPAYSFHVSADGQMQPVPFPHDALAGPGIPRHARQINTLSHGEVVCAVTISNPTRHVYTGGKGCVKIWDISQPGSKSPISQLDCLNRDNYIRSCKLLPDGRTLIVGGEASTLTIWDLASPTPRIKAELTSSAPACYALAISPDAKVCFSCCSDGNIAVWDLHNQTLVRQFQGHTDGASCIDISHDGTKLWTGGLDNTVRSWDLREGRQLQQHDFTSQIFSLGYCPTGEWLAVGMESSNVEVLHHTKPDKYQLHLHESCVLSLKFAYCGKWFVSTGKDNLLNAWRTPYGASIFQSKESSSVLSCDISADDKYIVTGSGDKKATVYEVIY

SEQ ID NO: 37 - protein, 773 aa, Homo sapiens
MIRDLSKMYPQTRHPAPHQPAQPFKFTISESCDRIKEEFQFLQAQYHSLKLECEKLASEKTEMQRHYVMYYEMSYGLNIEMHKQAEIVKRLNAICAQVIPFLSQEHQQQVVQAVERAKQVTMAELNAIIGQQLQAQHLSHGHGLPVPLTPHPSGLQPPAIPPIGSSAGLLALSSALGGQSHLPIKDEKKHHDNDHQRDRDSIKSSSVSPSASFRGAEKHRNSADYSSESKKQKTEEKEIAARYDSDGEKSDDNLVVDVSNEDPSSPRGSPAHSPRENGLDKTRLLKKDAPISPASIASSSSTPSSKSKELSLNEKSTTPVSKSNTPTPRTDAPTPGSNSTPGLRPVPGKPPGVDPLASSLRTPMAVPCPYPTPFGIVPHAGMNGELTSPGAAYAGLHNISPQMSAAAAAAAAAAAYGRSPVVGFDPHHHMRVPAIPPNLTGIPGGKPAYSFHVSADGQMQPVPFPPDALIGPGIPRHARQINTLNHGEVVCAVTISNPTRHVYTGGKGCVKVWDISHPGNKSPVSQLDCLNRDNYIRSCRLLPDGRTLIVGGEASTLSIWDLAAPTPRIKAELTSSAPACYALAISPDSKVCFSCCSDGNIAVWDLHNQTLVRQFQGHTDGASCIDISNDGTKLWTGGLDNTVRSWDLREGRQLQQHDFTSQIFSLGYCPTGEWLAVGMENSNVEVLHVTKPDKYQLHLHESCVLSLKFAHCGKWFVSTGKDNLLNAWRTPYGASIFQSKESSSVLSCDISVDDKYIVTGSGDKKATVYEVIY

SEQ ID NO: 38 - protein, 1273 aa, Homo sapiens [sequence omitted]

SEQ ID NO: 39 - protein, 51 aa, Artificial Sequence
  derived from NKX2.2
FSKAQTYELERRFRQQRYLSAPEREHLASLIRLTPTQVKIWFQNHRYKMKR

SEQ ID NO: 40 - DNA, 10 bp, Artificial Sequence
  DNA binding site of NKX2.2
tnaagtnntt